(19) United States
(12) Patent Application Publication
(10) Pub. No.: US 2021/0116466 A1
(43) Pub. Date: Apr. 22, 2021

(54) METHOD AND SYSTEM FOR EVALUATING RISK OF AGE-RELATED MACULAR DEGENERATION

(71) Applicants: Renatech Co., Ltd., Isehara-shi, Kanagawa (JP); Juntendo Educational Foundation, Tokyo (JP)

(72) Inventors: Seiichi Inagaki, Isehara-shi, Kanagawa (JP); Naoyuki Okamoto, Isehara-shi, Kanagawa (JP); Takenori Inomata, Tokyo (JP)

(73) Assignees: Renatech Co., Ltd., Isehara-shi, Kanagawa (JP); Juntendo Educational Foundation, Tokyo (JP)

(21) Appl. No.: 16/500,530

(22) PCT Filed: Apr. 4, 2018
(86) PCT No.: PCT/JP2018/014382

§ 371 Date: Oct. 3, 2019

(30) Foreign Application Priority Data
Apr. 4, 2017 (JP) 2017-074848

(51) Int. Cl. G01N 33/84; G16H 50/30; G01N 33/49

(52) U.S. Cl. CPC G01N 33/84; G01N 33/49; G16H 50/30

(57) ABSTRACT

An AMD risk evaluation method is provided. The concentrations of a set of evaluation elements contained in a serum sample 2 taken from a subject are measured (step S1), the concentration data of the set of evaluation elements thus measured are applied to a predetermined discriminant function to perform an operation (step S2); and whether or not the subject suffers from AMD is discriminated based on the operation result obtained by applying the concentration data to the discriminant function (step S3). The discrimination is carried out in accordance with the concentration balance (pattern) of the set of evaluation elements. The set of evaluation elements is designated by choosing all or part of specific elements that have the concentration data for both of the case group and the control group based on the discriminant abilities in arbitrary combinations of the specific elements.

### TECHNICAL FIELD

The present invention relates to a method and a system for evaluating the risk of age-related macular degeneration and more particularly, to a risk evaluation method of age-related macular degeneration that utilizes the concentration balance of elements (correlations among the concentrations of a set of evaluation elements) contained in a human serum, and a risk evaluation system used for this method.

### BACKGROUND ART

Age-related macular degeneration (which may also be termed AMD hereinafter) is a disease that the tissue called macula that plays an important role when a person looks at anything is changed by damages with age to result in a visual impairment.

AMD is not uncommon in the Western countries: however, in recent years, the number of patients with AMD in Japan is in an increasing trend according to the Westernization of diet. As the disease that causes blindness halfway through his/her life in Japan, glaucoma has been ranked in the first place and diabetic retinopathy has been ranked in the second one since before. However, in recent years, the number of the patients with AMD has been increasing rapidly and now, AMD is ranked in the fourth place. AMD develops due to the occurrence of something wrong in the “macula” existing at the center of the eye's retina by aging. The macula, which is located at the center of the retina and to which important cells that govern the eyesight are concentrated, has a function of discriminating a large part of optical information, such as the shape, size, color, depth, and distance of things, When the macula is impaired, symptoms, such as the central part of a thing looks distorted, the central part of a thing becomes invisible, and the eyesight degrades, will appear. Most of patients with AMD are 50 or older and the number of male patients is about three times as many as that of the female patients.

It has been said that various living environments, such as smoking, sunlight, excessive drinking, and insufficient vitamins are related to the cause of AMD in addition to aging. In particular, it has been clarified by the researches conducted in the Western countries that smoking is a dangerous factor for AMD. In addition, it has been suggested that trace elements are also related to. For example, in Non-Patent Literature 1, it is reported that in the aqueous humor the concentrations of Cd, Co, Fe, and Zn are higher, the concentration of Cu is lower, and the concentrations of Mg and Se are the same compared with the ordinary persons without AMD. In Non-Patent Literature 2, it is reported that within the trace elements contained in the blood Pb, Hg, and Cd have negative relevance to AMD, and Mg and Zn have positive relevance to AMD. In Non-Patent Literature 3, it is reported that accumulation of Fe is seen and the concentration of Zn is lower with respect to the patients with AMD. From these reports, it is assumed that some deep relationship exists between the onset of AMD and the trace elements.

On the other hand, Patent Literature 1 discloses a cancer evaluation method that utilizes the correlations between the onset of cancer and the concentrations of elements contained in a human serum. This method, which was developed by one of the applicants of the present application, comprises the correlation operating step of operating a correlation among concentrations of a set of evaluation elements contained in a serum which is taken from a subject by applying concentration data of the set of evaluation elements to a discriminant function for discriminating which of a case group and a control group the subject belongs to; and the indicator obtaining step of obtaining an indicator for indicating whether or not the subject suffers from any type of cancer based on the correlation operated in the correlation operating step. In this method, as the set of evaluation elements, a combination of 7 elements of S, P, Mg, Zn, Cu, Ti, and Rb or a combination of 16 elements of Na, Mg, Al, P, K, Ca, Ti, Mn, Fe, Zn, Cu, Se, Rb, Ag, Sn, and S is chosen. This method have advantageous effects that the risk of suffering cancer of a subject can be estimated with high accuracy, the disadvantages of early degeneration and high cost that arise in the case where in-blood amino acid concentrations are utilized do not occur, and this method can be applied easily to group or mass examinations. (See Claims 1 and 2, Paragraphs 0036, 0057-0061, 0070-0074, and FIGS. 1 and 14.)

### PRIOR ART LITERATURE

### Patent Literature

[Patent Literature 1] Japanese Examined Patent Publication No. 5,470,848

### Non-Patent Literature

[Non-Patent Literature 1] Junemann A G et al., Levels of aqueous humor trace elements in patients with non-exsudative age-related macular degeneration: a case-control study. PLoS ONE. 2013; 8(2): e56734
[Non-Patent Literature 2] Park S J et al., Five heavy metallic elements and age-related macular degeneration: Korean National Health and Nutrition Examination Survey, 2008-2011. Ophthalmology, 2015 January; 122(1): 129-37
[Non-Patent Literature 3] Ugarte M et al., Iron, zinc, and copper in retinal physiology and disease, Sury Ophthalmol 2013 November-December; 58(6): 585-609

### SUMMARY OF THE INVENTION

### Problems to be Resolved by the Invention

As described above, it is estimated from the reports of Non-Patent Literatures 1 to 3 that some deep relationship exists between the onset of AMD and the trace elements. Accordingly, the inventors found the possibility that makes it possible to estimate the risk of suffering from AMD by knowing the correlations among the in-serum concentrations of a specific set of elements based on the information estimated from the reports of Non-Patent Literatures 1 to 3 and the findings obtained from the development process of the cancer evaluation method disclosed in Patent Literature 1; thereafter, the inventors created the present invention.

Accordingly, an object of the present invention is to provide an AMD risk evaluation method and an AMD risk evaluation system that make it possible to estimate the risk of suffering from AMD of a subject with high accuracy and that do not have the disadvantages of early degeneration after sampling and high cost that arise in the case where the in-blood amino acid concentrations are utilized.

Another object of the present invention is to provide an AMD risk evaluation method and an AMD risk evaluation system that can be easily applied to group or mass examinations.

The other objects not specifically mentioned will become clear to those skilled in the art from the following description and drawings attached.

### Means for Solving the Problems

(1) According to the first aspect of the present invention, an AMD risk evaluation method is provided, which comprises:

the correlation operating step of operating a correlation among concentrations of a set of evaluation elements contained in a serum which is taken from a subject by applying concentration data of the set of evaluation elements to a discriminant function for discriminating which of a case group and a control group the subject belongs to; and

the indicator obtaining step of obtaining an indicator for discriminating whether or not the subject suffers from AMD based on the correlation operated in the correlation operating step;

wherein the set of evaluation elements is designated by choosing all or part of specific elements that have the concentration data for both of the case group and the control group based on the discriminant abilities in arbitrary combinations of the specific elements.

With the AMD risk evaluation method according to the first aspect of the present invention, as explained above, the concentration data of the set of evaluation elements contained in the serum which is taken from the subject are applied to the discriminant function for discriminating which of the case group and the control group the subject belongs to, thereby operating the correlation among the concentrations of the set of evaluation elements in the serum and then, the indicator for discriminating whether or not the subject suffers from AMD is obtained based on the correlation thus obtained. Moreover, the set of evaluation elements is designated by choosing all or part of the specific elements that have the concentration data for both of the case group and the control group (in other words, the concentrations were measurable for both of the case group and the control group) based on the discriminant abilities in arbitrary combinations of the specific elements. Accordingly, the risk of suffering from AMD of the subject can be estimated with high accuracy and at the same time, the disadvantages of early degeneration and high cost that arise in the case where the in-blood amino acid concentrations are utilized do not occur.

Furthermore, after obtaining the concentration data of the set of evaluation elements in the serum which is taken from the subject, which of the case group and the control group the subject belongs to can be discriminated by automatic operation using a computer. Accordingly, the discrimination can be performed easily and quickly even if the number of the subjects is large, which means that this method is easily applicable to group or mass examinations.

(2) In a preferred embodiment of the AMD risk evaluation method according to the first aspect of the present invention, the set of evaluation elements is designated by choosing all of the specific elements.
(3) In another preferred embodiment of the AMD risk evaluation method according to the first aspect of the present invention, the set of evaluation elements is designated by choosing part of the specific elements using a stepwise method.
(4) In still another preferred embodiment of the AMD risk evaluation method according to the first aspect of the present invention, the set of evaluation elements is designated by choosing one of the arbitrary combinations of the specific elements whose discriminant ability is equal to or larger than a desired value.
(5) In a further preferred embodiment of the AMD risk evaluation method according to the first aspect of the present invention, a set of 15 elements of Na, Mg, P, S, K, Ca, Fe, Cu, Zn, Se, Rb, Sr, As, Mo, and Cs is designated as the set of evaluation elements.
(6) In a further preferred embodiment of the AMD risk evaluation method according to the first aspect of the present invention, a set of 5 elements of S, Ca, Rb, As, and Cs is designated as the set of evaluation elements.
(7) In a further preferred embodiment of the AMD risk evaluation method according to the first aspect of the present invention, a set of 17 elements of Na, Mg, P, S, K, Ca, Fe, Cu, Zn, As, Sr, Rb, Se, Mo, Ni, Co, and Li is designated as the set of evaluation elements.
(8) In a further preferred embodiment of the AMD risk evaluation method according to the first aspect of the present invention, a set of 6 elements of S, K, Ca, Fe, Se, and Mo is designated as the set of evaluation elements.
(9) In a further preferred embodiment of the AMD risk evaluation method according to the first aspect of the present invention, the preliminary examination step of conducting a preliminary examination of the serum prior to obtaining the concentration data of the set of evaluation elements in the serum is further provided;

wherein the set of evaluation elements is designated by the preliminary examination step.

(10) According to the second aspect of the present invention, an AMD risk evaluation system is provided, which comprises:

a data storage section for storing concentration data of a set of evaluation elements contained in a serum which is taken from a subject;

a discriminant function generation section for generating a discriminant function for discriminating which of a case group and a control group the subject belongs to; and

an evaluation result operation section for operating a correlation among concentrations of the set of evaluation elements contained in the serum by applying the concentration data of the subject stored in the data storage section to the discriminant function generated by the discriminant function generation section, thereby outputting an evaluation result that discriminates whether or not the subject suffers from AMD based on the correlation;

wherein the set of evaluation elements is designated by choosing all or part of specific elements that have the concentration data for both of the case group and the control group based on the discriminant abilities in arbitrary combinations of the specific elements.

With the AMD risk evaluation system according to the second aspect of the present invention, as explained above, the concentration data of the set of evaluation elements contained in the serum which is taken from the subject are applied to the discriminant function for discriminating which of the case group and the control group the subject belongs to, thereby operating the correlation among the concentrations of the set of evaluation elements in the serum and then, an evaluation result that discriminates whether or not the subject suffers from AMD is obtained based on the correlation thus obtained. Moreover, the set of evaluation elements is designated by choosing all or part of the specific elements that have the concentration data for both of the case group and the control group (in other words, the concentrations were measurable for both of the case group and the control group) based on the discriminant abilities in arbitrary combinations of the specific elements. Accordingly, the risk of suffering from AMD of the subject can be estimated with high accuracy and at the same time, the disadvantages of early degeneration and high cost that arise in the case where the in-blood amino acid concentrations are utilized do not occur.

Furthermore, after obtaining the concentration data of the set of evaluation elements in the serum which is taken from the subject, which of the case group and the control group the subject belongs to can be discriminated by automatic operation using a computer. Accordingly, the discrimination can be performed easily and quickly even if the number of the subjects is large, which means that this system is easily applicable to group or mass examinations.

(11) In a preferred embodiment of the AMD risk evaluation system according to the second aspect of the present invention, the set of evaluation elements is designated by choosing all of the specific elements.
(12) In another preferred embodiment of the AMD risk evaluation system according to the second aspect of the present invention, the set of evaluation elements is designated by choosing part of the specific elements using a stepwise method.
(13) In still another preferred embodiment of the AMD risk evaluation system according to the second aspect of the present invention, the set of evaluation elements is designated by choosing one of the arbitrary combinations of the specific elements whose discriminant ability is equal to or larger than a desired value.
(14) In a further preferred embodiment of the AMD risk evaluation system according to the second aspect of the present invention, a set of 15 elements of Na, Mg, P, S, K, Ca, Fe, Cu, Zn, Se, Rb, Sr, As, Mo, and Cs is designated as the set of evaluation elements.
(15) In a further preferred embodiment of the AMD risk evaluation system according to the second aspect of the present invention, a set of 5 elements of S, Ca, Rb, As, and Cs is designated as the set of evaluation elements.
(16) In a further preferred embodiment of the AMD risk evaluation system according to the second aspect of the present invention, a set of 17 elements of Na, Mg, P, S, K, Ca, Fe, Cu, Zn, As, Sr, Rb, Se, Mo, Ni, Co, and Li is designated as the set of evaluation elements.
(17) In a further preferred embodiment of the AMD risk evaluation system according to the second aspect of the present invention, a set of 6 elements of S, K, Ca, Fe, Se, and Mo is designated as the set of evaluation elements.
(18) In a further preferred embodiment of the AMD risk evaluation system according to the second aspect of the present invention, a preliminary examination section for conducting a preliminary examination of the serum prior to obtaining the concentration data of the set of evaluation elements in the serum is further provided; wherein the set of evaluation elements is designated by the preliminary examination.

### Advantageous Effects of the Invention

With the AMD risk evaluation method according to the first aspect of the present invention and the AMD risk evaluation system according to the second aspect of the present invention, there are advantageous effects that the risk of suffering from AMD of a subject can be estimated with high accuracy, the disadvantages of early degeneration after sampling and high cost that arise in the case where the in-blood amino acid concentrations are utilized do not occur, and this method and this system can be applied easily to group or mass examinations.

### BRIEF DESCRIPTION OF THE DRAWINGS

FIG. 1 is a flowchart showing the basic principle of the AMD risk evaluation method according to the present invention.

FIG. 2 is a functional block diagram showing the basic structure of the AMD risk evaluation system according to the present invention.

FIG. 3 is a conceptual diagram showing the fact that the discrimination result about which of the control group and the case group the subjects belong to can be obtained by integrating the discrimination results for the respective specific elements in the AMD risk evaluation method according to the present invention.

FIG. 4 is a table showing the analysis result of the concentration data of the concentration-measurable elements contained in the serums (samples) of the 12 subjects in the case group and those of the 20 subjects in the control group, which is obtained by the AMD risk evaluation method according to the present invention, wherein the serums (samples) are subjected to a pretreatment using an acid.

FIG. 5 shows tables showing the result of discriminant analysis based on the analysis result of the concentration data of FIG. 4 (in which a pretreatment using an acid is applied), in which (a) shows the discrimination result in the case of using the sole concentration data of P, (b) shows the discrimination result in the case of using the sole concentration data of K, (c) shows the discrimination result in the case of using the sole concentration data of Fe, (d) shows the discrimination result in the case of using the sole concentration data of Se, (e) shows the discrimination result in the case of using the concentration data of 4 elements of P, K, Fe, and Se, (f) shows the discrimination result in the case of using the concentration data of all the 15 elements whose concentrations were measurable, and (g) shows the discrimination result in the case of using the concentration data of the elements chosen by the stepwise method.

FIG. 6 is an explanatory drawing showing an example of the discriminant formed based on the result of discriminant analysis of FIG. 5 (in which a pretreatment using an acid is applied), in which (a) shows the discriminant in the case where the concentration data of the 4 elements having significant differences between the case group and the control group are used, (b) shows the discriminant in the case where the concentration data of all the elements are used, and (c) shows the discriminant in the case where the concentration data of the elements chosen by a stepwise method are used.

FIG. 7 is a table showing the analysis result of the concentration data of the concentration-measurable elements contained in the serums (samples) of the 12 subjects in the case group and those of the 20 subjects in the control group, which is obtained by the AMD risk evaluation method according to the present invention, in which the serums (samples) are subjected to a pretreatment using an alkali.

FIG. 8A shows tables showing the result of discriminant analysis based on the analysis result of the concentration data of FIG. 7 (in which a pretreatment using an alkali is applied), in which (a) shows the discrimination result in the case of using the sole concentration data of Na, (b) shows the discrimination result in the case of using the sole concentration data of Mg, (c) shows the discrimination result in the case of using the sole concentration data of P, (d) shows the discrimination result in the case of using the sole concentration data of S, (e) shows the discrimination result in the case of using the sole concentration data of K, (f) shows the discrimination result in the case of using the sole concentration data of Ca, and (g) shows the discrimination result in the case of using the sole concentration data of Fe.

FIG. 8B shows tables showing the result of discriminant analysis based on the analysis result of the concentration data of FIG. 7 (in which a pretreatment using an alkali is applied), which is subsequent to FIG. 8A, in which (h) shows the discrimination result in the case of using the sole concentration data of Rb, (i) shows the discrimination result in the case of using the sole concentration data of Se, (j) shows the discrimination result in the case of using the sole concentration data of 9 elements of Na, Mg, P, S, K, Ca, Fe, Rb, and Se, (k) shows the discrimination result in the case of using the concentration data of all the 17 elements whose concentrations were measurable, and (I) shows the discrimination result in the case of using the concentration data of the elements chosen by the stepwise method.

FIG. 9 is an explanatory drawing showing an example of the discriminant formed based on the results of discriminant analysis of FIGS. 8A and 8B (in which a pretreatment using an alkali is applied), in which (a) shows the discriminant in the case where the concentration data of the 9 elements having significant differences between the case group and the control group are used, (b) shows the discriminant in the case where the concentration data of all the 17 elements are used, and (c) shows the discriminant in the case where the concentration data of the elements chosen by the stepwise method are used.

FIG. 10 is a table showing the measured concentration data of the concentration-measurable elements contained in the serums (samples) of the 12 subjects in the case group and those of the 20 subjects in the control group, which is obtained by the AMD risk evaluation method according to the present invention, in which the serums (samples) are subjected to a pretreatment using an acid.

FIG. 11 is a table showing the measured concentration data of the concentration-measurable elements contained in the serums (samples) of the 12 subjects in the case group and those of the 20 subjects in the control group, which is obtained by the AMD risk evaluation method according to the present invention, in which the serums (samples) are subjected to a pretreatment using an alkali.

FIG. 12 is a graph showing the result of risk evaluation of AMD using the discriminant score obtained by the AMD risk evaluation method according to the present invention, in which the serums (samples) used therein are subjected to a pretreatment using an acid.

FIG. 13 is a graph showing the result of risk evaluation of AMD using the discriminant score obtained by the AMD risk evaluation method according to the present invention, in which the serums (samples) used therein are subjected to a pretreatment using an alkali.

### EMBODIMENTS FOR CARRYING OUT THE INVENTION

Preferred embodiments of the present invention will be described below in detail while referring to the drawings attached.

### [Basic Principle of AMD Risk Evaluation Method of the Invention]

The inventors conducted research earnestly to develop a new AMD screening method that uses the concentrations (contents) of the elements contained in the serum of a subject and as a result, obtained the following findings: The first finding is that the risk of suffering from AMD seems to be able to be estimated based on the concentration change of the elements by comparing the concentrations of the elements contained in the serums of AMD patients and those of the elements contained in the serums of healthy persons (ordinary persons who were judged to have no AMD at the time of receiving a medical examination). The second finding is that Inductively-Coupled Plasma Mass Spectrometry (ICP-MS), which has been popularly used in the semiconductor fields, seems to be applicable to measuring the concentrations of the elements contained in the serums.

Accordingly, based on the aforementioned two findings, firstly, the inventors conducted a preliminary examination twice in order to designate (choose) the elements to be measured as “a set of evaluation elements” as shown below. In the first preliminary examination, a pretreatment using an acid or alkali was carried out and the elements to be measured were different form each other according to which of an acid and an alkali was used. For this reason, the case where a pretreatment using an acid is carried out and the case where a pretreatment using an alkali is carried out will be explained separately in the following:

[Case where Pretreatment Using Acid is Carried Out]

First Preliminary Examination: This is carried out to find the optimal measurement condition for measuring the elements contained in a serum. Here, first, a pretreatment using nitric acid was carried out. This pretreatment was to prevent difficulties in measuring the concentrations of the elements contained in a serum. The difficulties are, for example, that the concentration(s) of an element or elements is/are unable to be measured because the content(s) of an element or elements is/are close to the measurement limit of a concentration measuring apparatus used, and that measured values are not stable because the measured concentration value(s) of an element or elements fluctuate(s) widely in each measurement.

The aforementioned pretreatment is as follows: Specifically, 50 microliter (pi) of a serum sample was put into a container capable of sealing and then, a proper amount of a nitric acid solution and a proper amount of a hydrogen peroxide solution, each of which was concentration-adjusted, were added to the container, thereby mixing the serum sample with these solutions. Thereafter, the mixture thus formed was heated at a predetermined temperature for a predetermined period of time. In this way, proteins and amino acids contained in the serum sample were decomposed in order to prevent difficulties from occurring when measuring the concentrations of the elements contained in this serum sample. Following this, the mixture was diluted 500 times with pure water. In this way, a “serum sample for measurement” (a serum sample which the pretreatment was completed) was formed. On the other hand, a mixed standard solution for Inductively-Coupled Plasma Mass Spectrometry (ICP-MS) was appropriately diluted with a concentration-adjusted nitric acid solution, thereby forming calibration curves for the 9 elements of Fe, Cu, Zn, As, Sr, Rb, Se, Mo, and Cs. Moreover, single-element standard solutions, which were respectively prepared for the 6 elements of Na, Mg, P, S, K, and Ca, were mixed with each and appropriately diluted with a concentration-adjusted nitric acid solution, thereby forming calibration curves for the 6 elements of Na, Mg, P, S, K, and Ca. With these 15 calibration curves, the correlation coefficient of 0.9998 or higher was obtained for any of the corresponding 15 elements (which were determined by removing Ni, Co, and Li from the aforementioned 18 elements). In addition, calibration curves were formed for the excluded elements of Ni, Co, and Li also and then, the concentrations of these 3 elements were tried to be measured; however, the concentrations of them were unable to be measured stably and as a result, these 3 elements were excluded from the elements to be measured.

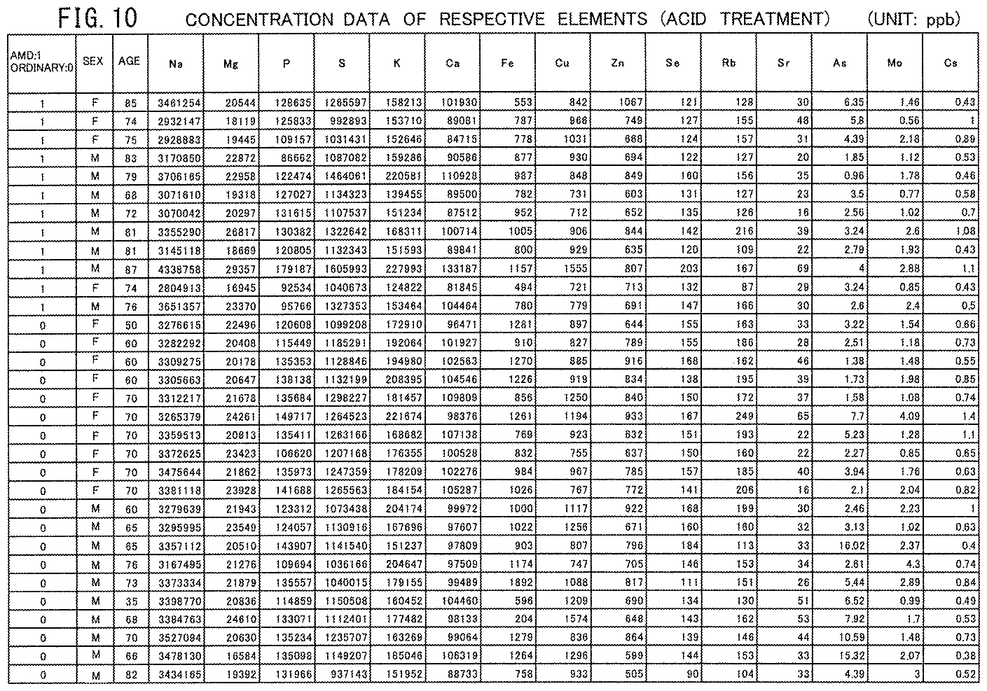

Furthermore, the aforementioned serum sample for measurement and internal standard solutions for ICP-MS were introduced into a known ICP-MS device in such a way that their flow rates were adjusted to have a predetermined flow rate ratio while supplying a predetermined high-frequency electric power to the device and at the same time, supplying a plasma gas, a nebulizer gas, and an auxiliary gas to the same device at appropriate flow rates. The internal standard solutions used here, which were four ones for Be, Te, Y, and Rh, were introduced into the same device in such a way that their flow rates were adjusted to have a predetermined flow rate ratio. In this way, the concentrations (contents) of the 18 elements of Na, Mg, P, S, K, Ca, Fe, Cu, Zn, Se, Rb, Sr, As, Mo, Cs, Ni, Co, and Li contained in the serum sample for measurement were measured. The reason why the elements to be measured were limited to these 18 ones is to choose elements whose concentrations were stably measurable when conducting the pretreatment using an acid (and a pretreatment using an alkali). When the concentrations were measured, the measurement condition was slightly changed. As a result, it was turned out that the measured concentration values of Ni, Co, and Li were unstable and therefore, these 3 elements were excluded from the elements to be measured; accordingly, the concentration data of the remaining 15 elements (Na, Mg, P, S, K, Ca, Fe, Cu, Zn, Se, Rb, Sr, As, Mo, and Cs) were obtained. An example of this result is shown in FIG. 10. The unit of the concentration is ppb in this figure. Based on the concentration data of these elements thus obtained, an optimal measurement condition was found.

To measure the concentrations of various elements, Inductively-Coupled Plasma Optical Emission Spectroscopy (ICP-OES), Inductively-Coupled Plasma Mass Spectroscopy (ICP-MS), Atomic Absorption Spectrometry (AAS), X-Ray Fluorescence analysis (XRF) and so on can be used in addition to ICP-MS. The reason why the inventors chose ICP-MS is that ICP-MS is recognized to be the simplest way where the quantitativity in measurement result is strict. Accordingly, if this condition is changed, or any other analyzing method that is more preferred is developed, it is needless to say that any other method than ICP-MS may be used for this purpose.

Second Preliminary Examination: This is carried out to determine the set of evaluation elements for concentration measurement. Under the optimal condition found in the first preliminary examination, the concentrations (contents) of the aforementioned 18 elements contained in the 20 serums (the serum samples for measurement) that belong to the control group and those of the same 18 elements contained in the 12 serums (the serum samples for measurement) that belong to the case group, which were the same as used in the first preliminary examination, were measured using ICP-MS. Thereafter, the difference of the in-serum concentrations of the aforementioned 18 elements between the case group and the control group was analyzed statistically.

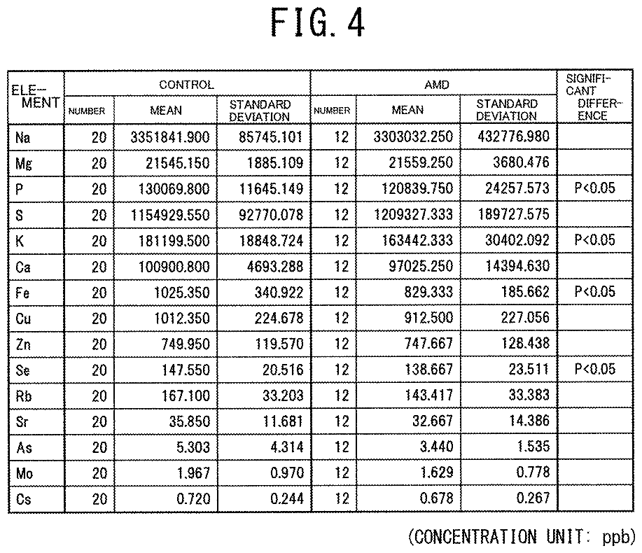

In the first preliminary examination, the elements having their measured concentration values (concentration data) with respect to all the subjects (all the serum samples for measurement), in other words, both of the subjects in the control group and those in the case group, were 15 elements of Na, Mg, P, S, K, Ca, Fe, Cu, Zn, Se, Rb, Sr, As, Mo, and Cs, excluding Ni, Co, and Li. For this reason, the measured concentration values of these 15 elements were analyzed statistically. This means that the 15 elements of Na, Mg, P, S, K, Ca, Fe, Cu, Zn, Se, Rb, Sr, As, Mo, and Cs were chosen as the elements to be analyzed statistically. In this statistical analysis, the test (t test, Welch's test) of the difference between the mean values (measured values, ranking) and discriminant analysis (the simultaneous method and the stepwise method) were used. The result of data analysis is shown in FIG. 4. As seen from FIG. 4, significant differences (i.e., p<0.05) were observed with respect to only 4 elements of P, K, Fe, and Se.

Subsequently, about the 4 elements of P, K, Fe, and Se that significant differences were observed, discriminant analysis was carried out in the case of using the sole concentration data of each of these 4 elements and then, discriminant analysis was carried out again in the case of using the concentration data of all of these 4 elements. Moreover, similar discriminant analysis was carried out in the case of using all of the concentration data of the 15 elements of Na, Mg, P, S, K, Ca, Fe, Cu, Zn, Se, Rb, Sr, As, Mo, and Cs that had their measured values (concentration data) in both the control group and the case group, in other words, with respect to all the subjects (all the serum samples for measurement), in which the simultaneous method was used. Furthermore, similar discriminant analysis was carried out again in the case of using the 5 elements of S, Ca, Rb, As, and Cs which were chosen from the aforementioned 15 elements by the stepwise method. In these discriminant analyses, in order to clarify the elements that relates to the difference between the case group and the control group with respect to each of the aforementioned 4 elements (P, K, Fe, and Se), discriminant analysis and the multiple logistic model were used. At that time, the combination that maximizes the difference between these two groups was sought while taking the combinations of the elements into consideration.

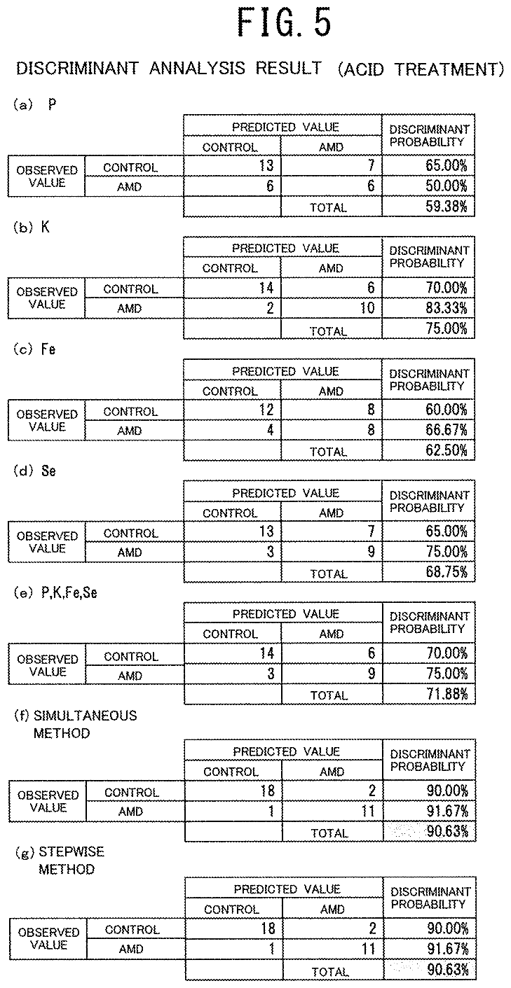

As a result, the discrimination results shown in FIG. 5(a) to FIG. 5(g) were obtained. FIG. 5(a), FIG. 5(b), FIG. 5(c), and FIG. 5(d) show the discrimination results in the cases where the sole concentration data of the 4 elements of P, K, Fe, and Se were respectively used. FIG. 5(e) shows the discrimination result in the case where all of the concentration data of the 4 elements of P, K, Fe, and Se were used in combination. FIG. 5(f) and FIG. 5(g) show respectively the discrimination results in the case of using all of the concentration data of the aforementioned 15 elements of Na, Mg, P, S, K, Ca, Fe, Cu, Zn, Se, Rb, Sr, As, Mo, and Cs, (where the simultaneous method was used), and in the case of using all of the concentration data of the aforementioned 5 elements (S, Ca, Rb, As, and Cs) which were chosen from these 15 elements by the stepwise method.

As seen from FIG. 5(a) to FIG. 5(g), the discriminant probability (the discrimination ability) in the case where the sole concentration data of the four elements of P, K, Fe, and Se were respectively used is about 60 to 75%, and the discriminant probability in the case where all of the concentration data of the four elements of P, K, Fe, and Se were used in combination is 71.88%; which means that the discrimination results in these two cases do not provide a high degree of effectiveness (high-level discriminant ability). However, as seen from FIG. 5(f), the discriminant probability in the case where all of the concentration data of the aforementioned 15 elements were used (the simultaneous method) has a high value of 90.63%, which means that an evaluation result with high accuracy can be expected. Moreover, as seen from FIG. 5(g), the discriminant probability in the case where all of the concentration data of the aforementioned 5 elements of S, Ca, Rb, As, and Cs were used (the stepwise method) also has a high value of 90.63%, which means that an evaluation result with high accuracy can be expected in this case also.

Accordingly, it was found that the case group and the control group can be discriminated with high accuracy by choosing one of (E1) the combination of 15 elements of Na, Mg, P, S, K, Ca, Fe, Cu, Zn, Se, Rb, Sr, As, Mo, and Cs, and (E2) the combination of 5 elements of S, Ca, Rb, As, and Cs, designating the combination thus chosen as the “set of evaluation elements”, measuring the in-serum concentrations of the “set of evaluation elements” (the concentrations of the serum samples for measurement) with respect to an individual subject, and statistically analyzes the in-serum concentrations thus measured. Thus, it was made apparent that a new method for evaluating (diagnosing) the presence or absence of the onset of AMD of a humans can be developed.

As described above, with the AMD risk evaluation method according to the present invention, by conducting the aforementioned two preliminary examinations, the “set of evaluation elements” can be designated by choosing a set of elements whose concentrations are to be measured from all the elements contained in all the serums (all the serum samples for measurement) of subjects. Therefore, taking the case where the aforementioned 15 elements (Na, Mg, P, S, K, Ca, Fe, Cu, Zn, Se, Rb, Sr, As, Mo, and Cs) whose concentrations were measured are chosen as the “set of evaluation elements” as an example, the details of analysis about the individual serum sample for measurement in the AMD risk evaluation method according to the present invention will be explained below.

First, discriminant analysis was carried out for the control group and the case group about the concentration data of the aforementioned 15 elements in the serums (the serum samples for measurement), which were designated as the “set of evaluation elements”. Concretely speaking, a test (t-test) for the difference between the population means of the control group and the case group was carried out. This was to search what degree these 15 elements affect the discrimination between these two groups. The result of this test is shown in FIG. 4.

Next, a discriminant function was obtained in the following way. This was to analyze the concentration balance (correlations) among the aforementioned 15 elements as the “set of evaluation elements”. The concentrations of the individual elements included personal differences and were difficult to be used as an indicator; therefore, the correlations of the concentrations among the elements were obtained here.

A discriminant function can be expressed in the following equation (1).

Discriminant Value (D)=Function (F) (Explanatory Variables 1 to n, Discriminant Coefficients) (1)
(n is an integer equal to or greater than 2.)

Taking the weight (the influence on discrimination) of the respective explanatory variables 1 to n into consideration, the equation (1) can be rewritten as the following equation (2).

Discriminant Value (D)=(Discriminant Coefficient 1)×(Explanatory Variable 1)+(Discriminant Coefficient 2)×(Explanatory Variable 2)+ . . . (Discriminant Coefficient n)×(Explanatory Variable n)+Constant (2)

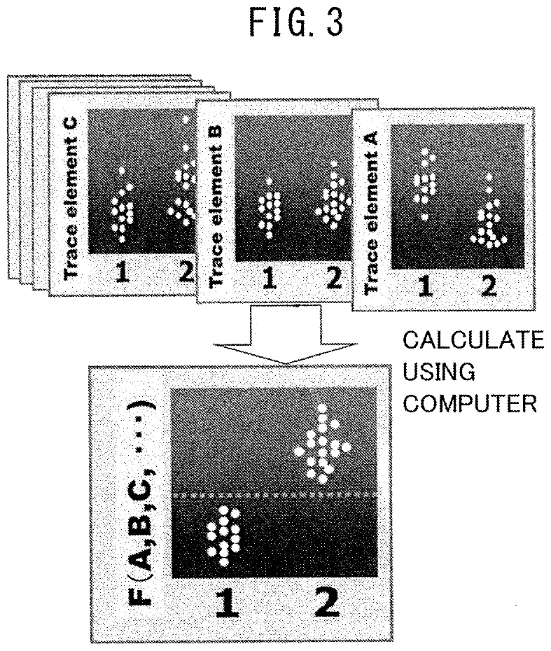

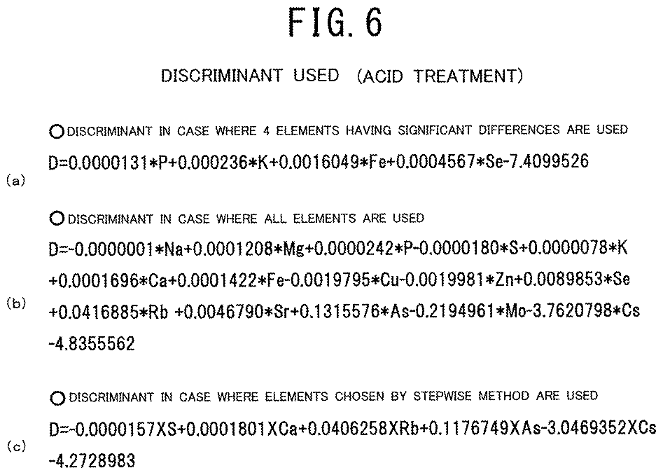

Here, based on the result (see FIG. 4) of the test (t-test) for the difference between the population means of the two groups, the aforementioned 15 elements (Na, Mg, P, S, K, Ca, Fe, Cu, Zn, Se, Rb, Sr, As, Mo, and Cs) which were chosen as the “set of evaluation elements” are defined as the explanatory variables and at the same time, the discriminant coefficients are used as the weight for these explanatory variables, resulting in a discriminant function. A desired discriminant function can be easily obtained by inputting the concentration values (concentration data) of these 15 elements into a known discriminant analysis program (e.g., SAS, SPSS, or the like) (see FIG. 3). Concretely, the discriminant function is given by an example of FIG. 6(b). In addition, even if any of discriminant analysis, multiple regression analysis, and logistic analysis was used, the discriminant function thus derived was expressed as the aforementioned equation (2).

By inputting the concentration data of the aforementioned 15 element into the discriminant function thus obtained, the discriminant value (discriminant score) (D) can be obtained. If the discriminant value (discriminant score) (D) calculated in this way is equal to or less than a predetermined reference value which is equal to or less than 0, it is judged that the subject belongs to the case group and that “the AMD acquiring risk is high”. On the other hand, if the discriminant value (D) is equal to or greater than a predetermined reference value which is equal to or greater than 0, it is judged that the subject belongs to the control group and that “the AMD acquiring risk is low”.

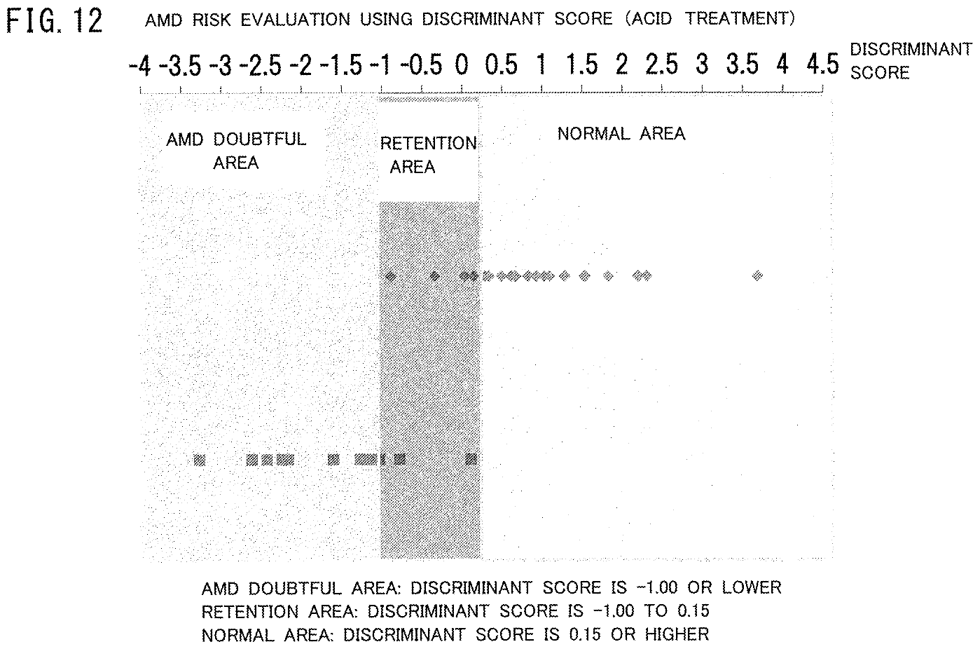

Here, the same serum samples for measurement as those used in the aforementioned two preliminary examinations are used and the concentrations of the aforementioned “set of evaluation elements” contained in these serum samples are measured; thereafter, the concentration data of the respective elements thus obtained are inputted into the discriminant function shown in FIG. 6(b). As a result, the discriminant value (discriminant score) (D) can be obtained. When which of the case group and the case group the individual subject belongs to is discriminated using the discriminant value thus obtained, the subject can be discriminated at a high discriminant probability of 90.63%, as shown in FIG. 5(f). A graphical expression of this discrimination result (evaluation result) is shown in FIG. 12. As seen from FIG. 12, if the discriminant value (discriminant score) (D) is equal to or less than the predetermined reference value which is equal to or less than 0 (the reference value is −1.00 in FIG. 12), it is evaluated that the subject belongs to the “AMD doubtful area” and that “the AMD acquiring risk is high”. On the other hand, if the discriminant value (D) is equal to or greater than the predetermined reference value which is equal to or greater than 0 (the reference value is +0.15 in FIG. 12), it is evaluated that the subject belongs to the “normal area” and that “the AMD acquiring risk is low”. if the discriminant value (D) is between the aforementioned two reference values (the reference values are −1.00 and +0.15 in FIG. 12), it is evaluated that the subject belongs to the “retention area” and that “the follow-up observation is necessary”.

Next, according to the necessity, to obtain the probability that the subject belongs to the case group or the control group, analysis is carried out using the multiple logistic model, thereby obtaining the incidence. The incidence is generally given by the following equation (3) using the discriminant value (D) which is obtained in the aforementioned discriminant analysis.

Incidence=1/[1+exp(−Discriminant Value)] (3)

Since the incidence can be given using the equation (3), the probability that the subject belongs to the case group also can be obtained. This means that the individual subject can know not only whether or not the AMD acquiring risk is high but also his/her own current AMD acquiring risk using the value (probability).

In addition, in the case where the aforementioned 5 elements (S, Ca, Rb, As, and Cs) are chosen and designated as the “set of evaluation elements” (in the case of using the stepwise method) instead of the aforementioned 15 elements also, the same result is obtained. As shown in FIG. 5(g), the subject can be discriminated at a high discriminant probability of 90.63%, which is the same as the case where the aforementioned 15 elements are used. as the “set of evaluation elements”. The discriminant function for this case is shown in FIG. 6(c). Furthermore, the discriminant function for the case where the aforementioned 4 elements (P, K, Fe, and Se) that have significant differences are used is shown in FIG. 6(a). In this case, the discriminant probability is 71.88%, which is fairly lower than those in the cases where the aforementioned 15 elements or the aforementioned 5 elements are chosen and designated.as the “set of evaluation elements”.

[Case where Pretreatment Using Alkali is Carried Out]

Next, the case where a pretreatment using an alkali is carried out will be explained below:

First Preliminary Examination: To find the optimal measurement condition for measuring the elements contained in a serum, a pretreatment using tetramethylammonium hydroxide (TMAH) was carried out. This pretreatment was to prevent difficulties in measuring the concentrations of elements contained in a serum like the aforementioned pretreatment using an acid.

The aforementioned pretreatment is as follows: Specifically, 100 microliter (pi) of a serum sample was put into a container capable of sealing and then, a proper amount of an aqueous solution that contains a TMAH solution, ethylenediaminetetraacetic acid, and triton X-100 at predetermined concentrations was added to the container, thereby diluting the serum sample 20 times. This is to decompose proteins and amino acids contained in the serum sample, thereby preventing difficulties from occurring when measuring the concentrations of elements contained in this sample. In this way, a “serum sample for measurement” (a serum sample which the pretreatment was completed) was formed. In addition, internal standard solutions for Inductively-Coupled Plasma Mass Spectrometry (ICP-MS) were added to the “serum sample for measurement” thus formed. The internal standard solutions used here, which were respectively prepared for Be, Te, Y, and Rh, were added to the same sample in such a way that their flow rates were adjusted to have a predetermined flow rate ratio. On the other hand, a mixed standard solution for ICP-MS was appropriately diluted with an aqueous solution that contains TMAH, ethylenediaminetetraacetic acid, and triton X-100 at predetermined concentrations, thereby forming calibration curves for the 11 elements of Fe, Cu, Zn, As, Sr, Co, Rb, Se, Mo, Ni, and Li. Moreover, single-element standard solutions, which were respectively prepared for the 7 elements of Na, Mg, P, S, K, Ca, and Cs were mixed with each and appropriately diluted with a concentration-adjusted TMAH solution and an aqueous solution that contains ethylenediaminetetraacetic acid and triton X-100 at predetermined concentrations, thereby forming calibration curves for the 7 elements of Na, Mg, P, S, K, Ca, and Cs. With these 17 calibration curves, the correlation coefficient of 0.9998 or higher was obtained for any of the corresponding 17 elements (which were determined by removing Cs from the aforementioned 18 elements). In addition, a calibration curve was formed for the excluded element of Cs also and then, the concentration of this element was tried to be measured; however, the concentration of the element Cs was unable to be measured stably and as a result, this element was excluded from the elements to be measured.

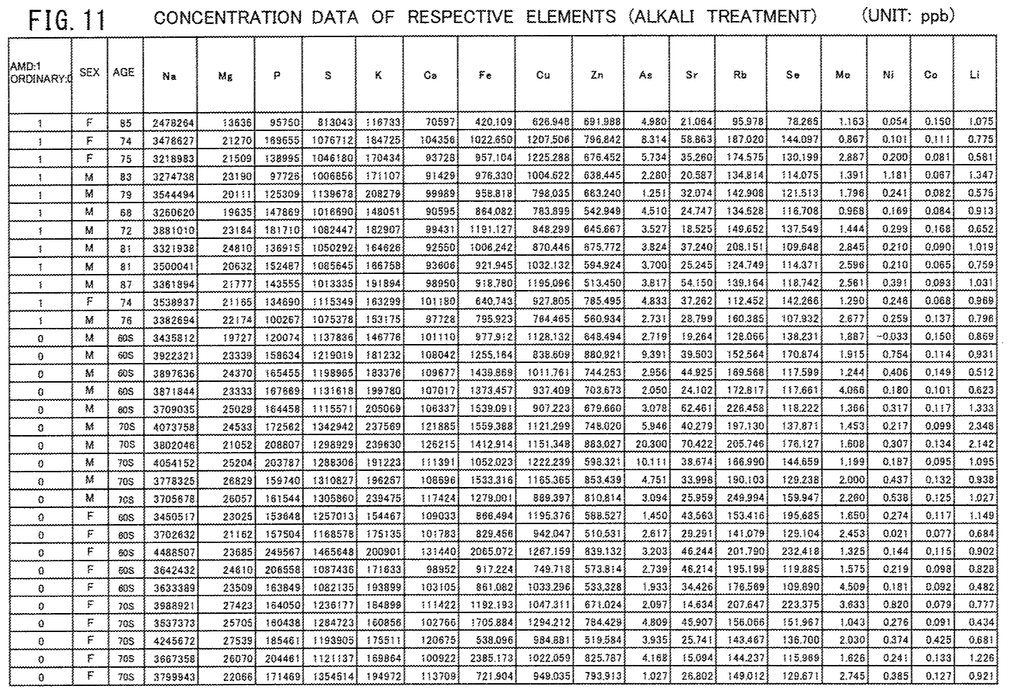

Furthermore, the aforementioned serum sample for measurement (to which the internal standard solutions for ICP-MS was added) was introduced into a known ICP-MS device in such a way that its flow rate was adjusted to have a predetermined value while supplying a predetermined high-frequency electric power to the device and at the same time, supplying a plasma gas, a nebulizer gas, and an auxiliary gas to the same device at appropriate flow rates. Here, the internal standard solutions for ICP-MS were already added to the serum sample for measurement. The internal standard solutions used here, which were four ones prepared for Be, Te, Y, and Rh, were already added to the serum sample for measurement and therefore, unlike the aforementioned pretreatment using an acid, it is unnecessary to separately introduce them into the device concurrently with the aforementioned serum sample for measurement. In this way, the concentrations (contents) of the 18 elements of Na, Mg, P, S, K, Ca, Fe, Cu, Zn, Se, Rb, Sr, As, Mo, Cs, Ni, Co, and Li contained in the serum sample for measurement were measured. The reason why elements to be measured were limited to these 18 ones is the same as that of the aforementioned pretreatment using an acid. When the concentrations were measured, the measurement condition was slightly changed. As a result, it was turned out that the measured concentration value of one element of Cs was unstable and therefore, Cs was excluded from the elements to be measured. Accordingly, the concentration data of the remaining 17 elements (Na, Mg, P, S, K, Ca, Fe, Cu, Zn, Se, Rb, Sr, As, Mo, Ni, Co, and Li) were obtained. An example of this result is shown in FIG. 11. The unit of the concentration is ppb in this figure. Based on the concentration data of these elements thus obtained, an optimal measurement condition was found.

Second Preliminary Examination: This is carried out to determine the set of evaluation elements for concentration measurement. Under the optimal condition found in the first preliminary examination, the concentrations (contents) of the aforementioned 18 elements contained in the 20 serums (the serum samples for measurement) that belong to the control group and those in the 12 serums (the serum samples for measurement) that belong to the case group, which were the same as the control and case groups used in the first preliminary examination, were measured using ICP-MS. Thereafter, the difference of the concentrations of the aforementioned 18 elements contained in the serums of the case group and those of the control group thus obtained was analyzed statistically.

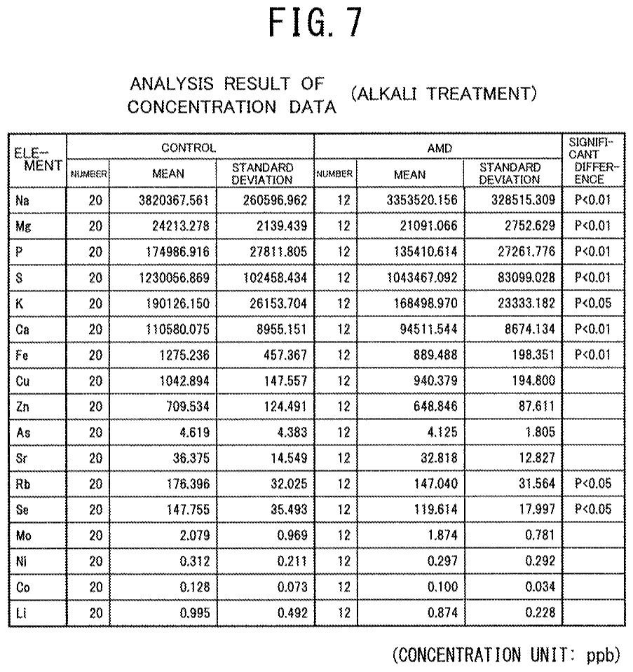

The elements having their measured concentration values (concentration data) with respect to all the subjects (all the serum samples for measurement), in other words, both of the subjects in the control group and those in the case group, were 17 elements of Na, Mg, P, S, K, Ca, Fe, Cu, Zn, Se, Rb, Sr, As, Mo, Ni, Co, and Li, excluding Cs. For this reason, the measured concentration values of these 17 elements were analyzed statistically. This means that these 17 elements were chosen as the elements to be analyzed statistically. The method used in this statistical analysis is the same as that of the aforementioned pretreatment using an acid. The result of data analysis is shown in FIG. 7. As seen from FIG. 7, significant differences (i.e., p<0.01) were observed with respect to only 9 elements of Na, Mg, P, S, K, Ca, Fe, Rb, and Se.

Subsequently, about the 9 elements that significant differences were observed, discriminant analysis was carried out in the case of using the sole concentration data of each of these 9 elements and then, discriminant analysis was carried out again in the case of using the concentration data of all of these 9 elements. Moreover, similar discriminant analysis was carried out in the case of using all of the concentration data of the 17 elements of Na, Mg, P, S, K, Ca, Fe, Cu, Zn, Se, Rb, Sr, As, Mo, Ni, Co, and Li, that had their measured values (concentration data) in both the control group and the case group, in other words, with respect to all the subjects (all the serum samples for measurement), in which the simultaneous method was used. Furthermore, similar discriminant analysis was carried out again in the case of using the 6 elements of S, K, Ca, Fe, Se, and Mo which were chosen from the aforementioned 17 elements by the stepwise method. In these discriminant analyses, in order to clarify the elements that relates to the difference between the case group and the control group with respect to each of the aforementioned 9 elements (Na, Mg, P, S, K, Ca, Fe, Rb, and Se), discriminant analysis and the multiple logistic model were used. At that time, the combination that maximizes the difference between these two groups was sought while taking the combinations of the elements into consideration.

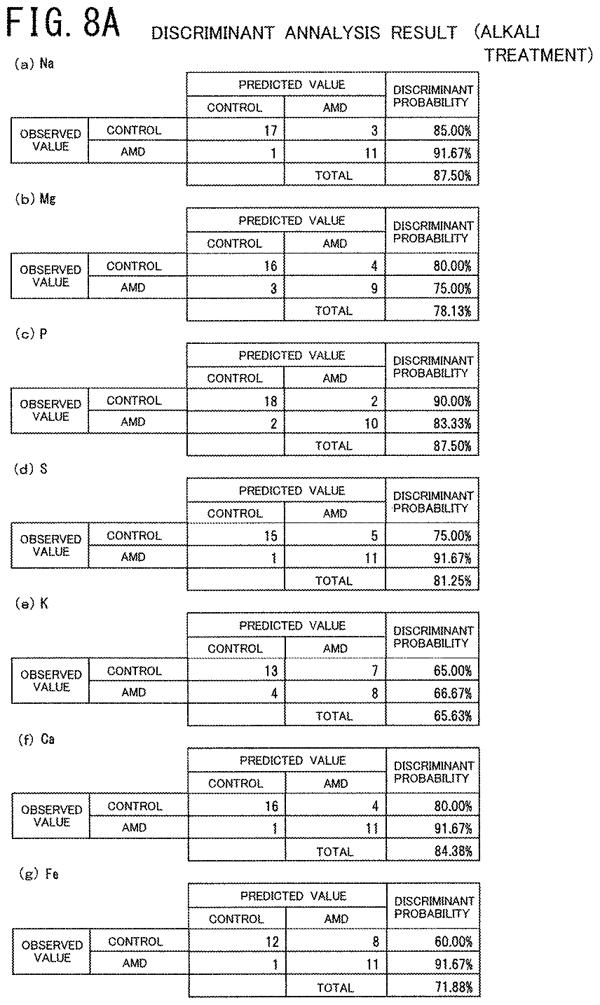

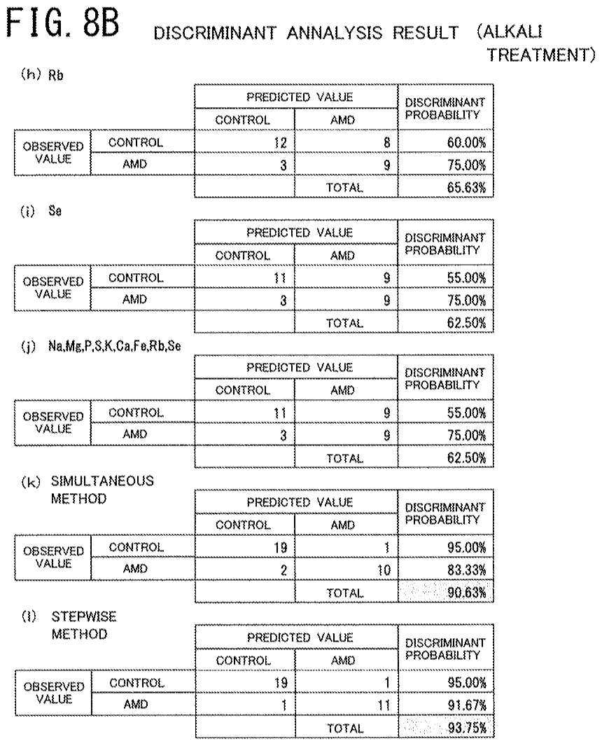

As a result, the discrimination results shown in FIG. 8A(a) to FIG. 8A(g) and FIG. 8B(h) to FIG. 8B(I) were obtained. FIG. 8A(a), FIG. 8A(b), FIG. 8A(c), FIG. 8A(d), FIG. 8A(e), FIG. 8A(f), FIG. 8A(g), FIG. 8A(h), and FIG. 8A(i) show the discrimination results in the cases where the sole concentration data of each of the 9 elements of Na, Mg, P, S, K, Ca, Fe, Rb, and Se were respectively used. FIG. 8B(j) shows the discrimination result in the case where all of the concentration data of the 9 elements of Na, Mg, P, S, K, Ca, Fe, Rb, and Se were used in combination. FIG. 8B(k) and FIG. 8B(I) show respectively the discrimination results in the case where all of the concentration data of the aforementioned 17 elements (Na, Mg, P, S, K, Ca, Fe, Cu, Zn, Se, Rb, Sr, As, Mo, Ni, Co, and Li) are used (where the simultaneous method was used) and in the case where all of the concentration data of the aforementioned 6 elements (S, K, Ca, Fe, Se, and Mo) which were chosen from these 17 elements by the stepwise method.

As seen from FIG. 8A(a) to FIG. 8A(g) and FIG. 8B(h) to FIG. 8B(i), the discriminant probability in the case where the sole concentration data of the 9 elements of Na, Mg, P, S, K, Ca, Fe, Rb, and Se were respectively used is about 60 to 87%, which is slightly higher than that of the aforementioned case using an acid. As seen from FIG. 8B(h) to FIG. 8B(j), the discriminant probability in the case where the concentration data of these 9 elements were used in combination is 62.50%, which is lower than that of the aforementioned case using an acid. In any of these two cases, the discrimination results do not provide a high degree of effectiveness (high-level discriminant ability). However, as seen from FIG. 8B(k), the discriminant probability in the case where all of the concentration data of the aforementioned 17 elements were used (the simultaneous method) has a high value of 90.63% in discrimination ability, which means that an evaluation result with high accuracy can be expected. Moreover, as seen from FIG. 8B(I), the discriminant probability in the case where all of the concentration data of the aforementioned 6 elements of S, K, Ca, Fe, Se, and Mo were used (the stepwise method). also has a high value of 93.75% higher than the case of the simultaneous method; which means that an evaluation result with high accuracy can be expected in this case also.

Accordingly, it was found that the case group and the control group can be discriminated with high accuracy by choosing one of (E3) the combination of 17 elements of Na, Mg, P, S, K, Ca, Fe, Cu, Zn, Se, Rb, Sr, As, Mo, Ni, Co, and Li and (E4) the combination of 6 elements of S, K, Ca, Fe, Se, and Mo, designating the combination thus chosen as the “set of evaluation elements”, measuring the in-serum concentrations of the “set of evaluation elements” (the concentrations of the serum samples for measurement) with respect to an individual subject; and statistically analyzes the in-serum concentrations thus measured. Thus, similar to the case of the pretreatment using an acid, it was made apparent that a new method for evaluating (diagnosing) the presence or absence of the onset of AMD of a human can be developed in the case of the pretreatment using an alkali also.

As described above, in the case of the pretreatment using an alkali also, similar to the case of the pretreatment using an acid, the “set of evaluation elements” can be designated by choosing a set of elements whose concentrations are to be measured from all the elements contained in all the serums (all the serum samples for measurement) of subjects. Then, by conducting a statistical analysis in the same way as used in the aforementioned case of the pretreatment using an acid using the “set of evaluation elements” thus designated, a discriminant value (discriminant score) (D) can be calculated. If the discriminant value (discriminant score) (D) calculated in this way is equal to or less than a predetermined reference value which is equal to or less than 0, it is judged that the subject belongs to the case group and that “the AMD acquiring risk is high”. On the other hand, if the discriminant value (D) is equal to or greater than a predetermined reference value which is equal to or greater than 0, it is judged that the subject belongs to the control group and that “the AMD acquiring risk is low”.

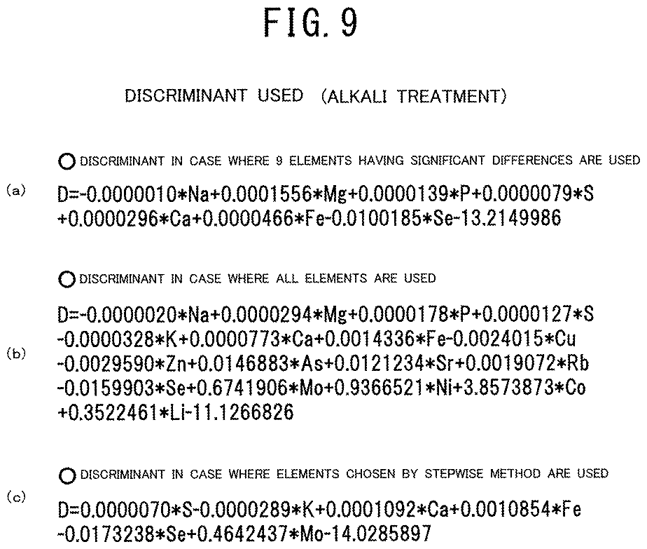

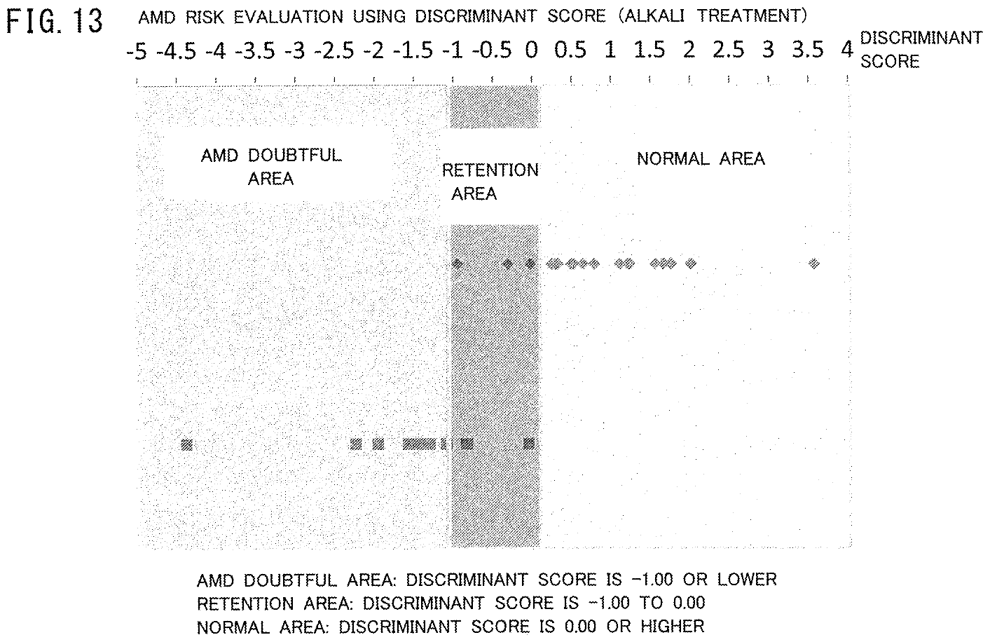

Here, the same serum samples for measurement as those used in the aforementioned two preliminary examinations are used and the concentrations of the aforementioned “set of evaluation elements” contained in these serum samples are measured; thereafter, the concentration data of the respective elements thus obtained are inputted into the discriminant function shown in FIG. 9(b). As a result, the discriminant value (discriminant score) (D) can be obtained. When which of the case group and the case group the individual subject belongs to is discriminated using the discriminant value thus obtained, the subject can be discriminated at a high discriminant probability of 90.63%, as shown in FIG. 8B(k). A graphical expression of this discrimination result (evaluation result) is shown in FIG. 13. As seen from FIG. 13, if the discriminant value (discriminant score) (D) is equal to or less than the predetermined reference value which is equal to or less than 0 (the reference value is −1.00 in FIG. 13), it is evaluated that the subject belongs to the “AMD doubtful area” and that “the AMD acquiring risk is high”. On the other hand, if the discriminant value (D) is equal to or greater than the predetermined reference value which is equal to or greater than 0 (the reference value is 0.00 in FIG. 13), it is evaluated that the subject belongs to the “normal area” and that “the AMD acquiring risk is low”. if the discriminant value (D) is between the aforementioned two reference values (the reference values are −1.00 and 0.00 in FIG. 13), it is evaluated that the subject belongs to the “retention area” and that “the follow-up observation is necessary”.

Thereafter, by carrying out an analysis using the multiple logistic model to calculate the “incidence” according to the necessity, the probability that the subject belongs to the case group also can be obtained. This means that the subject can know not only whether or not the AMD acquiring risk is high but also his/her own current AMD acquiring risk using the value (probability) with the AMD risk evaluation method according to the present invention.

In addition, in the case where the aforementioned 9 elements (Na, Mg, P, S, K, Ca, Fe, Se, and Rb) are chosen and designated as the “set of evaluation elements” (in the case of the stepwise method) instead of the aforementioned 17 elements also, the same result is obtained. As shown in FIG. 8B(I), similar to the case of choosing the aforementioned 17 elements, the subject can be discriminated at a high discriminant probability of 93.75%. The discriminant function for this case is shown in FIG. 9(c). Furthermore, the discriminant function for the case where the aforementioned 9 elements (Na, Mg, P, S, K, Ca, Fe, Se, and Rb) that have significant differences are used is shown in FIG. 9(a). In this case, the discriminant probability is 62.50%, which is fairly lower than those in the cases where the aforementioned 17 elements or the aforementioned 9 elements are chosen and designated.as the “set of evaluation elements”.

### (Process of AMD Risk Evaluation Method of Invention)

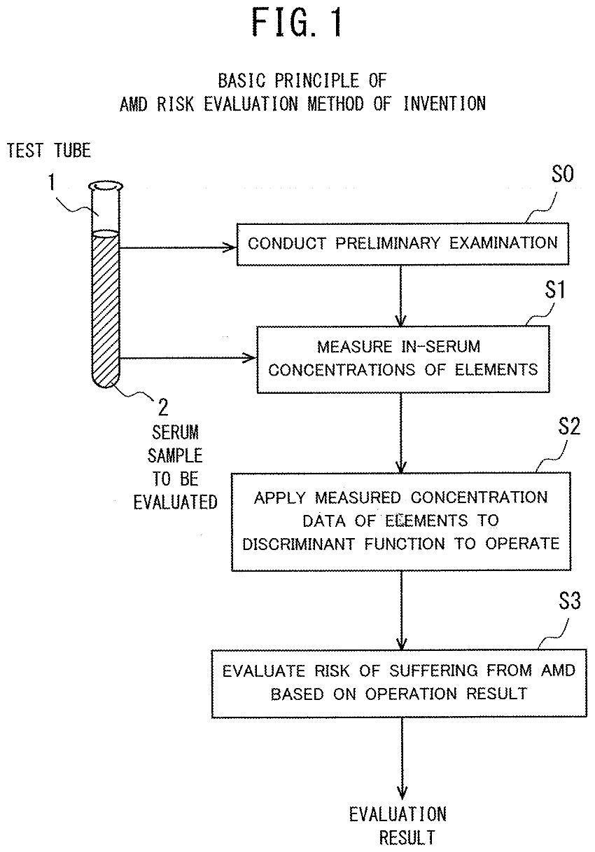

Next, the AMD risk evaluation method according to the present invention will be explained below with reference to FIG. 1.

With the AMD risk evaluation method according to the present invention, as clearly seen from FIG. 1, first, the aforementioned preliminary examinations (twice) are carried out (step S0). This step S0 may be termed the preliminary examination step. The preliminary examination step is a step for determining an optimum measuring condition of the element concentrations and for choosing and designating the “set of evaluation elements”, in which the latter is more important. Once the “set of evaluation elements” is designated, the execution of the step S0 is unnecessary and it is sufficient that only the steps S1 to S3 which will be explained later are carried out. It is sufficient that the preliminary examination(s) (step S0) is/are carried out each time a set of serum samples 2 taken from a predetermined number of subjects is sent.

Next, a serum sample 2 that has been collected from a subject is put into, for example, a test tube 1, and then, the test tube 1 is placed in a suitable analyzing apparatus (e.g., an ICP mass spectrometer) and analyzed, thereby measuring the concentrations of the predetermined elements (the set of evaluation elements) in the sample 2 (Step S1). As the set of evaluation elements whose concentrations are to be measured here, preferably, one of the aforementioned combinations (E1) to (E4) is used.

Next, the concentration data of the set of evaluation elements contained in the serum sample 2 obtained in the step S1 are applied to a predetermined discriminant function and an operation is conducted (step S2). As the discriminant function used here, for example, the discriminant function shown in FIG. 6(b) or that shown in FIG. 6(c) is chosen, or the discriminant function shown in FIG. 9(b) or that shown in FIG. 9(c) is chosen.

Finally, based on the operation result obtained in the step S2, whether or not the subject from which the serum sample 2 has been collected suffers from AMD is discriminated. As a result, as shown in FIG. 5 or FIGS. 8A and 8B, a desired evaluation result about the presence or absence of the onset of AMD is obtained (step S3).

With the AMD risk evaluation method according to the present invention, in this way, the concentration data of the set of evaluation elements contained in a serum which is taken from a subject are applied to a predetermined discriminant function, thereby operating a correlation among the concentrations of the set of evaluation elements in the serum and then, whether or not the subject suffers from AMD is discriminated based on the correlation among the concentrations of the set of evaluation elements thus obtained. Accordingly, the risk of suffering from AMD of the subject can be estimated with high accuracy and at the same time, the disadvantages of early degeneration and high cost that arise in the case where the in-blood amino acid concentrations are utilized do not occur.

Furthermore, after obtaining the concentration data of the set of evaluation elements in the serum which is taken from the subject, which of the case group and the control group the subject belongs to can be discriminated by automatic operation using a computer. Accordingly, this method is easily applicable to group or mass examinations.

### [Basic Structure of AMD Risk Evaluation System of Invention]

Next, the AMD risk evaluation system according to the present invention will be explained below.

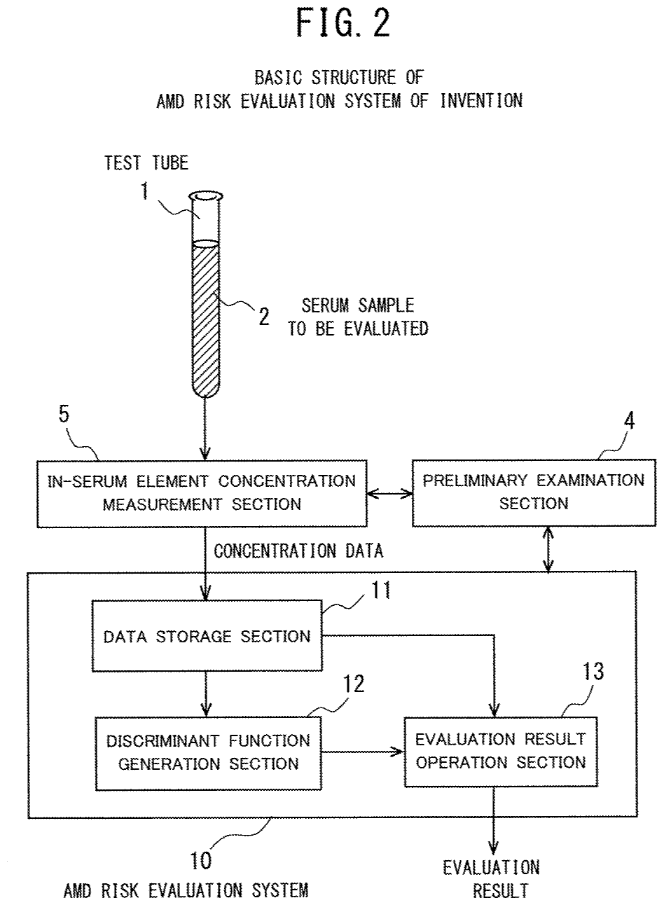

The basic structure of the AMD risk evaluation system 10 of the present invention is shown in FIG. 2. The AMD risk evaluation system 10, which is a system for carrying out the aforementioned AMD risk evaluation method of the present invention, comprises a data storage section 11, a discriminant function generation section 12, and an evaluation result operation section 13, as seen from FIG. 2.

A preliminary examination section 4 and an in-serum element concentration measurement section 5 are provided outside the AMD risk evaluation system 10.

The preliminary examination section 4 measures the in-serum concentrations of a set of evaluation elements using a serum that has been collected from a subject and that has been put into, for example, a test tube 1. The preliminary examination section 4 is a section for conducting the aforementioned preliminary examinations. In the preliminary examination section 4, a “set of evaluation elements” is chosen and designated by conducting the predetermined preliminary examinations. Thereafter, evaluation elements data corresponding to the set of evaluation elements thus designated is generated and sent to the in-serum element concentration measurement section 5. Here, the preliminary examinations are configured so as to be conducted using the in-serum element concentration measurement section 5, the discriminant function generation section 12, and the evaluation result operation section 13 which are explained later; however, the preliminary examinations may be configured so as to be conducted only in the preliminary examination section 4 by incorporating the same functions as described here into the preliminary examination section 4.

The in-serum element concentration measurement section 5 recognizes the set of evaluation elements to be measured using the evaluation elements data which is sent from the preliminary examination section 4. Then, this section 5 measures the concentrations of the set of evaluation elements contained in a serum sample 2. In this way, the in-serum concentration data of the set of evaluation elements which is obtained in the in-serum element concentration measurement section 5 is supplied to the data storage section 11. As the in-serum element concentration measurement section 5, for example, a known ICP mass spectrometer is used.

The data storage section 11 is a section for storing the concentration data of the set of evaluation elements obtained in the in-serum element concentration measurement section 5, which is usually formed by a known storage device. The data storage section 11 stores the concentration data of the set of evaluation elements contained in the serum collected from the subject.

The discriminant function generation section 12 is a section for generating a discriminant function that is explained above and that is used for the operation in the evaluation result operation section 13, which is usually formed to include a known program. The discriminant function generation section 12 generates a discriminant function for discriminating which of the case group and the control group the subject belongs to.

The evaluation result operation section 13 operates a correlation among the concentrations of the set of evaluation elements contained in the serum by applying the concentration data of the subject stored in the data storage section 11 to the discriminant function generated by the discriminant function generation section 12, thereby outputting an aforementioned evaluation result that discriminates whether or not the subject suffers from AMD based on the correlation thus operated. Based on the evaluation result thus outputted, the presence or absence of the onset risk of AMD for the subject is evaluated.

When the aforementioned AMD risk evaluation method according to the present invention is carried out with the AMD risk evaluation system 10, the onset risk of AMD is calculated using, for example, pattern analysis of the in-serum concentrations of the set of evaluation elements, and the result that the possibility of the onset of AMD is expressed stochastically based on the said risk is presented. Concretely speaking, serums (e.g., 0.5 cc) are collected at physical checkups which are conducted in medical institutions or diagnosis institutions and then, are subjected to concentration measurement of the set of specific evaluation elements at inspection agencies. Thereafter, based on the concentration data of the set of evaluation elements measured at the inspection agencies, the risk of suffering from AMD is calculated at an institution like, for example, a risk evaluation center (provisional name). The calculation result of the risk thus obtained is delivered to blood collection agencies and then, sent to a medical examinee from the blood collection agencies. If the examinee is suspected to suffer from AMD, the blood collection agencies recommend him/her to receive an “existing AMD examination”. The personal information is systemized so as not to reach the inspection agencies and the risk evaluation center through the encryption or consecutive numbering which is executed at the blood collection agencies.

The aforementioned embodiments and examples are exemplary embodied ones of the present invention. Thus, it is needless to say that the present invention is not limited to these embodiments and examples and any other modification is applicable to the embodiments and examples without departing the spirit of the invention.

For example, in the aforementioned embodiments, a pretreatment using an acid or an alkali is applied to the serum sample; however, it is needless to say that the present invention is not limited to these pretreatments. Any pretreatment other than those is available. Moreover, these pretreatments are not always necessary. If no difficulty occurs when measuring the element concentrations, such the pretreatments are unnecessary. The method of measuring the concentrations of the elements contained in a serum sample is optional; thus, the present invention is not limited to the methods or devices (ICP mass spectrometry, ICP mass spectrometry device) which are described in the aforementioned embodiments. If accurate concentration measurement of the elements contained in a serum sample is possible, any method and any device can be used for this purpose.

Furthermore, in the aforementioned embodiments, the elements to be concentration-measured are limited to 18 elements from the beginning; however, the present invention is not limited to these 18 elements. The kind and number of the elements to be concentration-measured before the set of evaluation elements is chosen and designated may be changed optionally.

### Example 1

Next, the present invention will be explained in more detail based on examples. Any of the following examples 1 to 4 corresponds to the AMD risk evaluation method according to the present invention.

Using the serums of the 20 subjects in the control group and those of the 12 subjects in the case group (32 subjects in total) that were subjected to the aforementioned pretreatment using an acid in the first preliminary examination as the serum samples 2, the concentrations (contents) of the 15 elements (Na, Mg, P, S, K, Ca, Fe, Cu, Zn, Se, Rb, Sr, As, Mo, and Cs) contained in these serums were measured by the ICP mass spectrometry. As a result, the result shown in FIG. 10 was obtained. In this example, these 15 elements were the “set of evaluation elements”. Thereafter, the difference of the concentrations of the set of evaluation elements thus obtained was analyzed statistically in the following way.

First, a test for the difference between the population means of the two groups (the control group and the case group) was carried out with respect to the serums (samples) of the 32 subjects and thereafter, the concentration data of the 15 elements (the set of evaluation elements) contained in the serums (samples) of the 32 subjects were subjected to discriminant analysis. The discriminant function shown in FIG. 6(b) (for the simultaneous method) was used here.

The final result of the discriminant analysis is shown in FIG. 5(f). As seen from this figure, 18 out of the 20 samples in the control group (healthy persons) were predicted to belong to the control group by the set of evaluation elements (Na, Mg, P, S, K, Ca, Fe, Cu, Zn, Se, Rb, Sr, As, Mo, and Cs) used in this discrimination and the remaining 2 samples were estimated to belong to the case group. In addition, 11 out of the 12 samples in the case group (AMD patients) were estimated to belong to the case group and the remaining 1 sample was estimated to belong to the control group. From this result, it was found that the discriminant ability was that the sensitivity (which indicates the rate of actual patients to be judged patients) was 91.7% (11/12) and the specificity (which indicates the rate of non-patients to be judged non-patients) was 90.0% (18/20).

It has been reported that the sensitivity is 59% and the specificity is 63% in the case of using a time-domain optical coherence tomograph (TD-OCT) as one of the current diagnostic imaging methods, and that the sensitivity is 901% and the specificity is 47% in the case of using a spectral-domain optical coherence tomograph (SD-OCT) as another of the current diagnostic imaging methods. Therefore, it is expected that the prediction (screening) method of suffering from AMD utilizing the difference between the concentration patterns of the specific in-serum elements, which was newly used here, will be a significant method.

### Example 2

The 5 elements of S, Ca, Rb, As, and Cs were used as the “set of evaluation elements”. The concentrations of these 5 elements were measured in the same way as used in Example 1 except that these 5 elements were used as the “set of evaluation elements”. Thereafter, the difference of the concentrations of the set of evaluation elements between the case group and the control group was analyzed statistically in the same way as EXAMPLE 1. The discriminant function shown in FIG. 6(c) (for the stepwise method) was used here. The final result of the discriminant analysis is shown in FIG. 5(g). As seen from this table, 18 out of the 20 samples in the control group (healthy persons) were predicted to belong to the control group by the set of evaluation elements (S, Ca, Rb, As, and Cs) used in this discrimination and the remaining 2 samples were estimated to belong to the case group. In addition, 11 out of the 12 samples in the case group (AMD patients) were estimated to belong to the case group and the remaining 1 sample was estimated to belong to the control group. From this result, it was found that the discriminant ability was that the sensitivity was 91.7% (11/12) and the specificity was 90.0% (18/20).

### Example 3

The pretreatment using an alkali (not an acid) was carried out in the first preliminary examination, and the 17 elements of Na, Mg, P, S, K, Ca, Fe, Cu, Zn, Se, Rb, Sr, As, Mo, Ni, Co, and Li were used as the “set of evaluation elements”. The concentrations of these 17 elements were measured in the same way as used in Example 1 except that these 17 elements were used as the “set of evaluation elements” and that the pretreatment using an alkali was carried out in the first preliminary examination and thus, the result shown in FIG. 11 was obtained. Thereafter, the difference of the concentrations of the set of evaluation elements between the case group and the control group was analyzed statistically in the same way as EXAMPLE 1. The discriminant function shown in FIG. 6(b) (for the simultaneous method) was used here. The final result of the discriminant analysis is shown in FIG. 8B(k). As seen from this figure, 19 out of the 20 samples in the control group (healthy persons) were predicted to belong to the control group by the set of evaluation elements (Na, Mg, P, S, K, Ca, Fe, Cu, Zn, Se, Rb, Sr, As, Mo, Ni, Co, and Li) used in this discrimination and the remaining 1 sample was estimated to belong to the case group. In addition, 10 out of the 12 samples in the case group (AMD patients) were estimated to belong to the case group and the remaining 2 samples were estimated to belong to the control group. From this result, it was found that the discriminant ability was that the sensitivity was 83.3% (10/12) and the specificity was 95.0% (19/20).

### Example 4

The 6 elements of S, K, Ca, Fe, Se, and Mo were used as the “set of evaluation elements”. The concentrations of these 9 elements were measured in the same way as used in Example 3 except that these 6 elements were used as the “set of evaluation elements”. Thereafter, the difference of the concentrations of the set of evaluation elements between the case group and the control group was analyzed statistically in the same way as EXAMPLE 1. The discriminant function shown in FIG. 9(c) (for the stepwise method) was used here. The final result of the discriminant analysis is shown in FIG. 8B(I). As seen from this figure, 19 out of the 20 samples in the control group (healthy persons) were predicted to belong to the control group by the set of evaluation elements (Na, Mg, P, S, K, Ca, Fe, Se, and Rb) used in this discrimination and the remaining 1 sample was estimated to belong to the case group. In addition, 11 out of the 12 samples in the case group (AMD patients) were estimated to belong to the case group and the remaining 1 sample was estimated to belong to the control group. From this result, it was found that the discriminant ability was that the sensitivity was 91.7% (11/12) and the specificity was 95.0% (19/20).

### INDUSTRIAL APPLICABILITY

The present invention is widely applicable to the fields where quick and convenient estimation of the presence or absence of the risk of suffering from AMD of humans (or animals) is expected.

### DESCRIPTION OF REFERENCE NUMERALS

1 test tube
2 serum sample
4 preliminary examination section
5 in-serum element concentration measurement section
10 cancer risk evaluation system
11 data storage section
12 discriminant function generation section
13 evaluation result operation section

1. An AMD risk evaluation method comprising:
the correlation operating step of operating a correlation among concentrations of a set of evaluation elements contained in a serum which is taken from a subject by applying concentration data of the set of evaluation elements to a discriminant function for discriminating which of a case group and a control group the subject belongs to; and
the indicator obtaining step of obtaining an indicator for discriminating whether or not the subject suffers from AMD based on the correlation operated in the correlation operating step;
wherein the set of evaluation elements is designated by choosing all or part of specific elements that have the concentration data for both of the case group and the control group based on the discriminant abilities in arbitrary combinations of the specific elements.

2. The AMD risk evaluation method according to claim 1, wherein the set of evaluation elements is designated by choosing all of the specific elements.

3. The AMD risk evaluation method according to claim 1, wherein the set of evaluation elements is designated by choosing part of the specific elements using a stepwise method.

4. The AMD risk evaluation method according to claim 1, wherein the set of evaluation elements is designated by choosing one of the arbitrary combinations of the specific elements whose discriminant ability is equal to or larger than a desired value.

5. The AMD risk evaluation method according to claim 1, wherein a set of 15 elements of Na, Mg, P, S, K, Ca, Fe, Cu, Zn, Se, Rb, Sr, As, Mo, and Cs is designated as the set of evaluation elements.

6. The AMD risk evaluation method according to claim 1, wherein a set of 5 elements of S, Ca, Rb, As, and Cs is designated as the set of evaluation elements.

7. The AMD risk evaluation method according to claim 1, wherein a set of 17 elements of Na, Mg, P, S, K, Ca, Fe, Cu, Zn, As, Sr, Rb, Se, Mo, Ni, Co, and Li is designated as the set of evaluation elements.

8. The AMD risk evaluation method according to claim 1, wherein a set of 6 elements of S, K, Ca, Fe, Se, and Mo is designated as the set of evaluation elements.

9. The AMD risk evaluation method according to claim 1, further comprising the preliminary examination step of conducting a preliminary examination of the serum prior to obtaining the concentration data of the set of evaluation elements in the serum;
wherein the set of evaluation elements is designated by the preliminary examination step.

10. An AMD risk evaluation system comprising:
a data storage section for storing concentration data of a set of evaluation elements contained in a serum which is taken from a subject;
a discriminant function generation section for generating a discriminant function for discriminating which of a case group and a control group the subject belongs to; and
an evaluation result operation section for operating a correlation among concentrations of the set of evaluation elements contained in the serum by applying the concentration data of the subject stored in the data storage section to the discriminant function generated by the discriminant function generation section, thereby outputting an evaluation result that discriminates whether or not the subject suffers from AMD based on the correlation;
wherein the set of evaluation elements is designated by choosing all or part of specific elements that have the concentration data for both of the case group and the control group based on the discriminant abilities in arbitrary combinations of the specific elements.

11. The AMD risk evaluation system according to claim 10, wherein the set of evaluation elements is designated by choosing all of the specific elements.

12. The AMD risk evaluation system according to claim 10, wherein the set of evaluation elements is designated by choosing part of the specific elements using a stepwise

13. The AMD risk evaluation system according to claim 10, wherein the set of evaluation elements is designated by choosing one of the arbitrary combinations of the specific elements whose discriminant ability is equal to or larger than a desired value.

14. The AMD risk evaluation system according to claim 10, wherein a set of 15 elements of Na, Mg, P, S, K, Ca, Fe, Cu, Zn, Se, Rb, Sr, As, Mo, and Cs is designated as the set of evaluation elements.

15. The AMD risk evaluation system according to claim 10, wherein a set of 5 elements of S, Ca, Rb, As, and Cs is designated as the set of evaluation elements.

16. The AMD risk evaluation system according to claim 10, wherein a set of 17 elements of Na, Mg, P, S, K, Ca, Fe, Cu, Zn, As, Sr, Rb, Se, Mo, Ni, Co, and Li is designated as the set of evaluation elements.

17. The AMD risk evaluation system according to claim 10, wherein a set of 6 elements of S, K, Ca, Fe, Se, and Mo is designated as the set of evaluation elements.

18. The AMD risk evaluation system according to claim 10, further comprising a preliminary examination section for conducting a preliminary examination of the serum prior to obtaining the concentration data of the set of evaluation elements in the serum;
wherein the set of evaluation elements is designated by the preliminary examination.
